(19) United States
(12) Patent Application Publication
(10) Pub. No.: US 2024/0081668 A1
(43) Pub. Date: Mar. 14, 2024

(54) BLOOD PRESSURE MEASURING DEVICE

(71) Applicant: CHARMCARE CO., LTD., Seoul (KR)

(72) Inventor: Dong Hwa LEE, Yongin-si (KR)

(21) Appl. No.: 18/518,325

(22) Filed: Nov. 22, 2023

Related U.S. Application Data
(63) Continuation of application No. PCT/KR2022/008009, filed on Jun. 7, 2022.

(30) Foreign Application Priority Data
Jun. 7, 2021 (KR) 10-2021-0073354

(51) Int. Cl. A61B 5/022; A61B 5/00

(52) U.S. Cl. CPC A61B 5/02233; A61B 5/6824; A61B 5/6831

(57) ABSTRACT

Provided is a blood pressure measuring device. The blood pressure measuring device is configured to be worn around a wrist of an examinee, and includes: a first strap configured to be wound on a part of the wrist adjacent to an ulnar artery; a second strap connected to the first strap to be worn around the wrist, configured to be worn on a part of the wrist adjacent to a radial artery, and including an air pocket into and from which air selectively flows; and a main body provided on the first strap or the second strap and including a measurement unit configured to measure a pressure of the air pocket and a controller configured to calculate a blood pressure of a user based on the pressure of the air pocket measured by the measurement unit.

### TECHNICAL FIELD

The present disclosure relates to a blood pressure measuring device, and more particularly to a blood pressure measuring device configured to be worn around a wrist of a user and to measure a blood pressure.

### BACKGROUND

Blood pressure information is one of important information to identify health conditions. The term “blood pressure” refers to the pressure of blood against the wall of a blood vessel. The pressure applied to the blood vessel when the heart contracts to push out blood is called “systolic blood pressure” or “maximal blood pressure”, and the pressure maintained in the blood vessel when the heart is relaxed to receive blood is called “diastolic blood pressure” or “minimal blood pressure”.

In general, a healthy person has a systolic blood pressure of less than 120 mmHg and a diastolic blood pressure of less than 80 mmHg Meanwhile, a person having high blood pressure has a systolic blood pressure of 140 mmHg or more and a diastolic blood pressure of 90 mmHg or more, and a person having low blood pressure has a systolic blood pressure of 90 mmHg or less and a diastolic blood pressure of 60 mmHg or less.

High blood pressure or low blood pressure can lead to health problems, and, thus, it is important to measure a blood pressure in order to obtain blood pressure information. Examples of methods of measuring a blood pressure may include a Korotkoff sounds method, an oscillometric method, a tonometric method, and the like.

The oscillometric method sufficiently presses a body part where arterial blood flows, so that the flow of the arterial blood can be stopped, and then, senses pulse waves, which are generated while the blood pressure is lowered at a constant speed or while the blood pressure of the body part where the arterial blood flows is raised at a constant speed, to measure a systolic pressure and a diastolic pressure.

Also, the tonometric method refers to a method of measuring a blood pressure based on the amplitude and shape of a pulse wave generated when a body part is compressed not to completely stop the flow of the arterial blood.

However, most of conventional devices for measuring a blood pressure of an examinee according to the oscillometric method are configured to be worn around an forearm of the examinee and to measure a blood pressure. However, the conventional blood pressure measuring devices configured to be worn around the forearm of the examinee and to measure the blood pressure are quite large to carry.

To solve this problem, Korean Patent No. 10-1349767 (hereinafter, referred to as a “prior art document”) discloses a wrist-mount blood pressure monitor configured to be wound around a wrist of an examinee to improve portability and equipped with an air chamber to compress an artery of the examinee. The wrist-mount blood pressure monitor of the prior art document is configured to measure a pressure of the air chamber when the air chamber compresses the artery of the examinee and to calculate a blood pressure of the examinee based on the measured pressure of the air chamber.

Meanwhile, as the amount of air filled in the air chamber increases, the accuracy in measuring the pressure of the air chamber increases. When the air chamber is not filled with a sufficient amount of air, an error may occur in measuring the pressure of the air chamber.

The prior art document discloses the air chamber that can be filled with air, but does not disclose the configuration of the air chamber to be filled with air as much as possible.

Also, as a contact area between the wrist and the air chamber filled with air and expanded increases, the artery of the examinee can be compressed more effectively. However, the air chamber disclosed in the prior art document expands like a balloon when filled with air, and, thus, the contact area between the air chamber and the wrist cannot be increased effectively.

### DISCLOSURE OF THE INVENTION

### Problems to be Solved by the Invention

The present disclosure is conceived to solve the problem of inaccurate calculation of a blood pressure caused by inaccurate measurement of a pressure of an air pocket of a blood pressure measuring device.

Also, the present disclosure is conceived to solve the problem of an insufficient contact area between a wrist and an air pocket when the air pocket of a blood pressure measuring device expands.

However, the problems to be solved by the present disclosure are not limited to the above-described problems, and other problems, which are not mentioned above, will be apparent to a person with ordinary skill in the art from the following description.

### Means for Solving the Problems

As a technical means for solving the problems, a blood pressure measuring device according to an embodiment of the present disclosure is configured to be worn around a wrist of an examinee, and includes: a first strap configured to be wound on a part of the wrist adjacent to an ulnar artery; a second strap connected to the first strap to be worn around the wrist, configured to be worn on a part of the wrist adjacent to a radial artery, and including an air pocket into and from which air selectively flows; and a main body provided on the first strap or the second strap and including a measurement unit configured to measure a pressure of the air pocket and a controller configured to calculate a blood pressure of a user based on the pressure of the air pocket measured by the measurement unit. The air pocket includes a compressing chamber which expands when filled with air and thus compresses the wrist and compresses the radial artery. The compressing chamber includes a first surface adjacent to the wrist and a second surface facing the first surface, and a part of the second surface protrudes toward the first surface and forms an expansion part.

Also, the compressing chamber may have one or more expansion parts.

Further, the second strap may have a plurality of holes formed along a longitudinal direction, and the air pocket may be equipped with an air pocket ring. The air pocket ring may be detachably coupled to any one of the plurality of holes to adjust a position of the air pocket on the second strap.

Furthermore, the main body may include a display unit configured to display the calculated blood pressure.

Moreover, the main body may include a communication unit configured to transmit and receive data to and from an external device.

As a technical means for solving the problems, a blood pressure measuring device according to another embodiment of the present disclosure is configured to be worn around a wrist of an examinee, and includes: a first strap configured to be wound on a part of the wrist adjacent to an ulnar artery; a second strap connected to the first strap to be worn around the wrist, configured to be worn on a part of the wrist adjacent to a radial artery, and including an air pocket into and from which air selectively flows; and a main body provided on the first strap or the second strap and including a measurement unit configured to measure a pressure of the air pocket and a controller configured to calculate a blood pressure of a user based on the pressure of the air pocket measured by the measurement unit. The air pocket includes a compressing chamber which includes a first surface adjacent to the wrist and a second surface facing the first surface and expands when filled with air and thus compresses the wrist with the first surface and compresses the radial artery. The air pocket also includes an auxiliary chamber which communicates with a part of the second surface of the compressing chamber and expands when filled with air, and has a cross-sectional diameter that increases and decreases in a direction parallel to the first surface of the compressing chamber and thus forms a concave part.

Also, the auxiliary chamber may have one or more concave parts.

Details of other embodiments will be included in the detailed description and the accompanying drawings.

### Effects of the Invention

According to the above-described means for solving the problems of the present disclosure, a blood pressure measuring device of the present disclosure is equipped with an air pocket including a compressing chamber having an expansion part. Therefore, it can be greatly expanded and thus can be filled with a large amount of air. Accordingly, it is possible to accurately measure a pressure of the air pocket and thus accurately calculate a blood pressure.

Also, the air pocket includes an auxiliary chamber having a concave part. Therefore, the air pocket expands into an elliptical shape, and, thus, a contact area between the air pocket and a wrist of an examinee can be effectively increased. Accordingly, it is possible to effectively compress an artery of the examinee and thus accurately measure a blood pressure.

### BRIEF DESCRIPTION OF THE DRAWINGS

FIG. 1 is a cross-sectional view of a blood pressure measuring device according to an embodiment of the present disclosure.

FIG. 2 is a diagram illustrating a blood pressure measuring device which is worn around a wrist of an examinee and in which an air pocket is filled with air.

FIG. 3A illustrates an expansion shape of a compressing chamber in which an expansion part is not formed.

FIG. 3B illustrates an expansion shape of a compressing chamber in which an expansion part is formed.

FIG. 4 illustrates an example of a method of manufacturing an auxiliary chamber which communicates with the compressing chamber.

FIG. 5A illustrates that an air pocket without an auxiliary chamber expands and compresses a wrist of an examinee.

FIG. 5B illustrates that an air pocket with an auxiliary chamber expands and compresses a wrist of an examinee.

FIG. 6 illustrates an example of a structure in which a first strap, a second strap, and an air pocket are connected.

FIG. 7 illustrates an example of a structure in which the air pocket of FIG. 6 is connected to a second strap.

FIG. 8 is a block diagram showing a main body of a blood pressure measuring device according to an embodiment of the present disclosure.

FIG. 9 illustrates an example of a main body equipped with a display unit.

### BEST MODE FOR CARRYING OUT THE INVENTION

Hereafter, embodiments will be described in detail with reference to the accompanying drawings so that the present disclosure may be readily implemented by a person with ordinary skill in the art. However, it is to be noted that the present disclosure is not limited to the embodiments but can be embodied in various other ways. In the drawings, parts irrelevant to the description are omitted for the simplicity of explanation, and like reference numerals denote like parts through the whole document.

Throughout this document, the term “connected to” may be used to designate a connection or coupling of one element to another element and includes both an element being “directly connected to” another element and an element being “electronically connected to” another element via another element.

Through the whole document, the term “on” that is used to designate a position of one element with respect to another element includes both a case that the one element is adjacent to the other element and a case that any other element exists between these two elements.

Further, through the whole document, the term “comprises or includes” and/or “comprising or including” used in the document means that one or more other components, steps, operation and/or existence or addition of elements are not excluded in addition to the described components, steps, operation and/or elements unless context dictates otherwise. Through the whole document, the term “about or approximately” or “substantially” is intended to have meanings close to numerical values or ranges specified with an allowable error and intended to prevent accurate or absolute numerical values disclosed for understanding of the present disclosure from being illegally or unfairly used by any unconscionable third party. Through the whole document, the term “step of” does not mean “step for”.

Hereafter, embodiments of the present disclosure will be described in detail with reference to the accompanying drawings and the following description. However, the present disclosure is not limited to the following embodiments and can be embodied in a different form. Like reference numerals generally denote like elements throughout the present specification.

Hereafter, a configuration of a blood pressure measuring device 1 according to an embodiment of the present disclosure will be described.

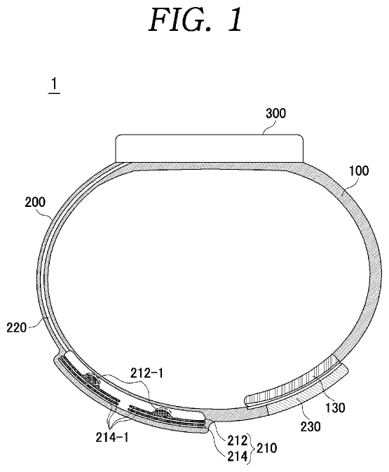

FIG. 1 is a cross-sectional view of a blood pressure measuring device according to an embodiment of the present disclosure.

Referring to FIG. 1, the blood pressure measuring device 1 according to an embodiment of the present disclosure may be configured to be worn around a wrist of an examinee, and may include a first strap 100 configured to be wound on a part of the wrist of the examinee, a second strap 200 configured to be worn on another part of the wrist of the examinee, and a main body 300 configured to calculate a blood pressure.

First, the first strap 100 will be described.

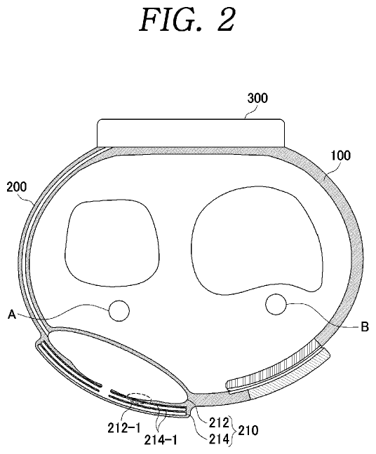

FIG. 2 is a diagram illustrating a blood pressure measuring device which is worn around a wrist of an examinee and in which an air pocket is filled with air.

Referring to FIG. 1 and FIG. 2, the first strap 100 may be placed to be worn on a part of the wrist of the examinee adjacent to an ulnar artery B and may be formed of a material having a predetermined strength or more to suppress bending.

For example, the first strap 100 may be formed of metal, plastic or hard rubber having a predetermined strength or more with elasticity.

Since the first strap 100 is formed of a material having a predetermined strength or more with elasticity, it is possible to suppress compression onto the wrist of the examinee and the ulnar artery B located inside the wrist by bending of the first strap 100. Therefore, it is possible to suppress stop of blood flowing in the ulnar artery B.

Therefore, the blood pressure measuring device 1 can measure a blood pressure according to the tonometric method by which a blood pressure can be measured while the flow of arterial blood is not completely stopped.

The first strap 100 may include a first connector 130 on one side, and the first connector 130 may be configured to be connected to the second strap 200 to be described later.

Hereafter, the second strap 200 will be described.

Referring to FIG. 1 and FIG. 2, the second strap 200 may be connected to the first strap 100 to be wound around the wrist of the examinee and may be placed to be worn on a part of the wrist of the examinee adjacent to a radial artery A. Also, the second strap 200 may include an air pocket 210, have an air flow path 220 therein, and include a second connector 230 on one side.

The air pocket 210 may be provided in the second strap 200 to compress a part of the wrist adjacent to the radial artery A of the examinee wearing the blood pressure measuring device 1, and air may selectively flow into and from the air pocket 210.

When the air pocket 210 is filled with air, the air pocket 210 expands and compresses the wrist and thus compresses the radial artery A as illustrated in FIG. 2. In this case, a force applied to the wrist by the air pocket 210 can be regulated by regulating the amount of air filled in the air pocket 210.

Meanwhile, the air pocket 210 may include a compressing chamber 212 which can compress the wrist and an auxiliary chamber 214 configured to communicate with the compressing chamber 212.

The compressing chamber 212 may have a chamber shape into and from which air flows, and may be formed of an elastic material, such as silicon, urethane, rubber, etc., which expands when filled with air and contracts when the air is exhausted.

Also, the compressing chamber 212 may include a first surface adjacent to the wrist and a second surface facing the first surface. A part of the second surface protrudes toward the first surface and forms an expansion part 212-1. The expansion part 212-1 may be formed into a cylindrical protrusion or a protrusion extending and protruding in a longitudinal direction, and is not limited to a specific shape.

One or more expansion parts 212-1 may be formed in the second surface of the compressing chamber 212, and may function to increase the volume of the compressing chamber 212 in order for the compressing chamber 212 to accommodate air as much as possible when air is filled in the compressing chamber 212.

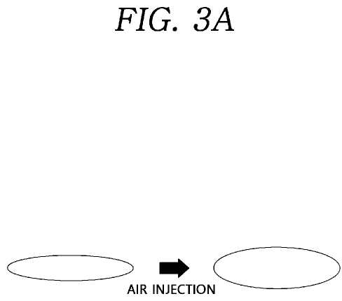

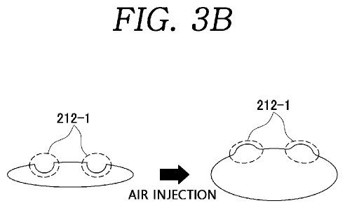

FIG. 3A illustrates an expansion shape of a compressing chamber in which an expansion part is not formed, and FIG. 3B illustrates an expansion shape of an compressing chamber in which an expansion part is formed.

For example, referring to FIG. 3A, when air is filled in the compressing chamber 212 in which the expansion part 212-1 is not formed, the compressing chamber 212 expands to a predetermined first volume.

Referring to FIG. 3B, when air is filled in the compressing chamber 212 in which the expansion part 212-1 is formed, the expansion part 212-1 expands outwards from the inside of the compressing chamber 212 as the compressing chamber 212 expands. Therefore, compared to the compressing chamber 212 in which the expansion part 212-1 is not formed, the compressing chamber 212 in which the expansion part 212-1 is formed can accommodate more air as much as the expansion part 212-1 expands.

The auxiliary chamber 214 may communicate with a part of the second surface of the compressing chamber 212, and may be formed of an elastic material, such as silicon, urethane, rubber, etc., which expands when filled with air and contracts when the air is exhausted.

Further, the auxiliary chamber 214 may have a cross-sectional diameter that increases and decreases in a direction parallel to the first surface of the compressing chamber 212 and thus form a concave part 214-1.

For example, referring to FIG. 1, the auxiliary chamber 214 may have a diameter that repeatedly increases and decreases in a direction away from the first surface of the compressing chamber 212 and thus form one or more concave parts 214-1.

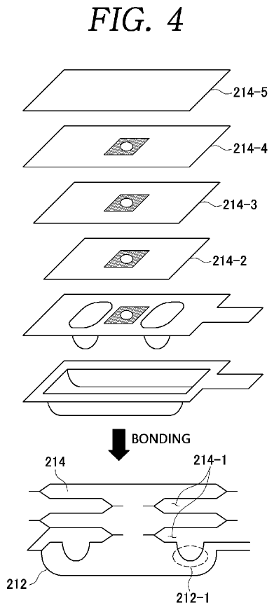

FIG. 4 illustrates an example of a method of manufacturing an auxiliary chamber which communicates with the compressing chamber.

For example, referring to FIG. 4, the auxiliary chamber 214 which communicates with the compressing chamber 212 and in which the concave part 214-1 is formed may be manufactured by sequentially stacking and bonding the first surface of the compressing chamber 212, the second surface having a first hole of the compressing chamber 212, a first plate 214-2 having a second hole of the auxiliary chamber 214, a second plate 214-3 having a third hole of the auxiliary chamber 214, a third plate 214-4 having a fourth hole of the auxiliary chamber 214, and a fourth plate 214-5 of the auxiliary chamber 214.

As described, the auxiliary chamber 214 connected to the compressing chamber 212 has the concave part 214-1, and, thus, when the compressing chamber 212 and the auxiliary chamber 214 are filled with air, the compressing chamber 212 may expand into an elliptical shape to increase a contact area between the air pocket and the wrist of the examinee.

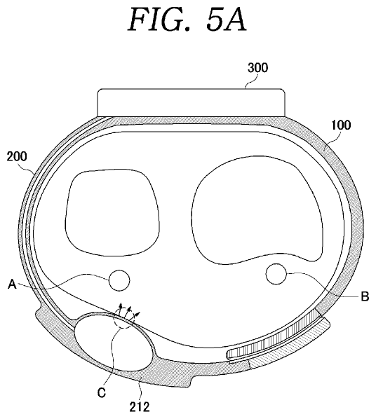

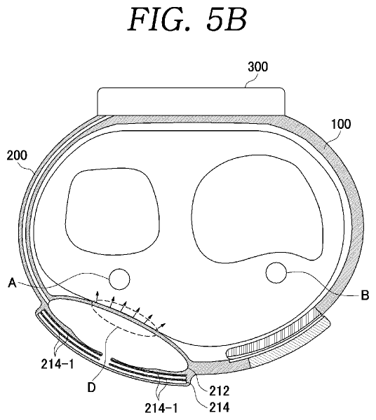

FIG. 5A illustrates that an air pocket without an auxiliary chamber expands and compresses a wrist of an examinee, and FIG. 5B illustrates that an air pocket with an auxiliary chamber expands and compresses a wrist of an examinee.

For example, referring to FIG. 5A, when the air pocket 210 without the auxiliary chamber 214 is filled with air, the air pocket 210 expands like a balloon, and, thus, a first contact area C between the air pocket 210 and the wrist of the examinee is decreased.

Referring to FIG. 5B, when the air pocket 210 with the auxiliary chamber 214 in which the concave part 214-1 is formed is filled with air, expansion of the compressing chamber 212 in a direction perpendicular to the first surface is suppressed by the auxiliary chamber 214 which predominantly expands in a direction parallel to the first surface of the compressing chamber 212. Therefore, the compressing chamber 212 expands into an elliptical shape, and a second contact area D between the air pocket 210 and the wrist of the examinee becomes greater than the first contact area C.

The air flow path 220 may be a flow path formed within the second strap 200 to enable air to flow into or from the air pocket 210. Specifically, the air flow path 220 may be connected to the compressing chamber 212 on one side and the main body 300 on the other side in order for air to flow into or from the air pocket 210 when the main body 300 operates.

The second connector 230 may be connected to the first connector 130 to connect the first strap 100 and the second strap 200.

For example, the first connector 130 and the second connector 230 may be formed of a conventional Velcro tape and thus can be attached to and detached from each other.

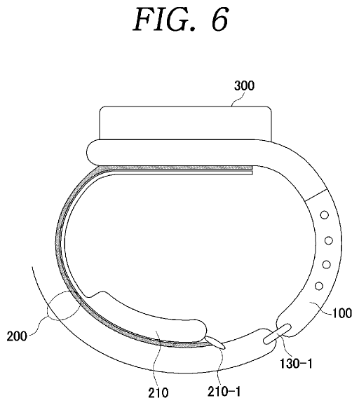

FIG. 6 illustrates an example of a structure in which a first strap, a second strap, and an air pocket are connected.

For example, referring to FIG. 6, the first connector 130 has a ring 130-1, and the second strap 200 formed of a Velcro tape is attached through the ring 130-1, and, thus, the first strap 100 can be connected to the second strap 200.

Further, the air pocket 210 may be equipped with an air pocket ring 210-1 and connected to the second strap 200.

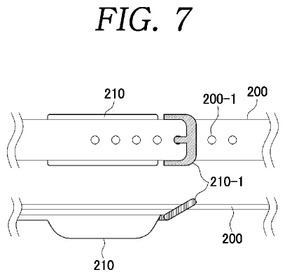

FIG. 7 illustrates an example of a structure in which the air pocket of FIG. 6 is connected to a second strap.

For example, referring to FIG. 7, the second strap 200 may have a plurality of holes 200-1 formed along a longitudinal direction, and the air pocket ring 210-1 may be configured to be detachably coupled to any one of the plurality of holes 200-1.

As described above, the air pocket ring 210-1 is configured to be selectively coupled to any one of the plurality of holes 200-1, and, thus, a position of the air pocket 210 can be adjusted to effectively compress the radial artery A of the examinee.

Hereafter, the main body 300 will be described.

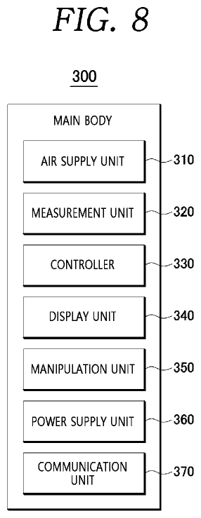

FIG. 8 is a block diagram showing a main body of a blood pressure measuring device according to an embodiment of the present disclosure.

Referring to FIG. 8, the main body 300 may control an operation of the blood pressure measuring device 1 of the present disclosure to calculate a blood pressure of the examinee, and may include an air supply unit 310, a measurement unit 320, a controller 330, a display unit 340, a manipulation unit 350, a power supply unit 360, and a communication unit 370.

The air supply unit 310 may supply air into the air pocket 210 of the second strap 200 through the air flow path 220 or suck air from the air pocket 210. The air supply unit 310 may be configured as a conventional air supply device including an air pump, a valve, etc.

The measurement unit 320 may function to measure a pressure of the air pocket 210 which is the basis of calculation of a blood pressure. The measurement unit 320 may be configured as a conventional pressure sensor or the like. For example, the measurement unit 320 may be configured as a pressure sensor in which the air flow path 220 is connected to the main body 300, and may measure a pressure of the air pocket 210.

The controller 330 may be configured as a conventional micro-computer, and may control operations of the components of the blood pressure measuring device 1. For example, the controller 330 may control an operation of the air supply unit 310 in order for the air pocket 210 to compress the wrist of the examinee with any pressure, and may control the display unit 340 to output specific information.

Further, the controller 330 may receive information about the pressure of the air pocket 210 measured by the measurement unit 320 and calculate a blood pressure of the examinee based on the received information. The controller 330 may also store blood pressure data of the examinee.

The display unit 340 may output information including characters or symbols to be seen by the user, such as the examinee, and may be configured including a conventional liquid crystal display LCD or the like.

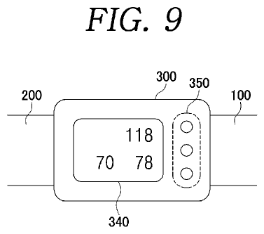

FIG. 9 illustrates an example of a main body equipped with a display unit.

For example, referring to FIG. 9, the display unit 340 may numerically display information about the blood pressure of the examinee calculated by the controller 330.

The manipulation unit 350 may be configured as a button or touch panel in order for the user, such as the examinee, to manipulate the blood pressure measuring device 1 by compressing or touching the button or touch panel with a predetermined force, and may be provided on an outer surface of the main body 300.

The power supply unit 360 may function to supply power to operate the blood pressure measuring device 1, and may be configured as a power supply means, such as a conventional battery or the like.

The communication unit 370 may be configured as a conventional communication module capable of transmitting and receiving data to and from an external device, and may utilize communication technologies, such as RFID (Radio Frequency Identification), UWB (Ultra Wide Band), Bluetooth, a wireless sensor network, to be suitable for data transmission and reception. The blood pressure measuring device 1 may transmit blood pressure data or distress signals to the external device through the communication unit 370.

Hereafter, the effects of the blood pressure measuring device of the present disclosure will be described in detail.

When the examinee wears the blood pressure measuring device 1 around the wrist and manipulates the manipulation unit 350, the air pocket 210 may compress the wrist adjacent to the radial artery A.

In this case, the first strap 100 is formed of a material having a predetermined strength or more to suppress compression onto the ulnar artery B. Therefore, it is possible to suppress stop of blood flowing in the ulnar artery B.

Further, the compressing chamber 212 of the air pocket 210 to be filled with air by manipulating the manipulation unit 350 includes the expansion part 212-1. Thus, the compressing chamber 212 can expand while effectively accommodating a large amount of air.

Furthermore, since the auxiliary chamber 214 having the concave part 214-1 is coupled to the second surface, the compressing chamber 212 may expand into an elliptical shape to increase a contact area between the air pocket and the wrist of the examinee when filled with air.

A pressure of the air pocket 210 applied to the wrist of the examinee may be measured by the measurement unit 320, and the controller 330 may calculate a blood pressure of the examinee based on information about the measured pressure of the air pocket 210.

Information about the calculated blood pressure of the examinee may be output to the display unit 340 or transmitted to an external device through the communication unit 370.

As described above, a blood pressure measuring device according to the present disclosure is equipped with an air pocket including a compressing chamber having an expansion part. Therefore, it can be greatly expanded and thus can be filled with a large amount of air. Accordingly, it is possible to accurately measure a pressure of the air pocket and thus accurately calculate a blood pressure.

Also, the air pocket includes an auxiliary chamber having a concave part. Therefore, the air pocket expands into an elliptical shape, and, thus, a contact area between the air pocket and a wrist of an examinee can be effectively increased. Accordingly, it is possible to effectively compress an artery of the examinee and thus accurately measure a blood pressure.

The above description of the present disclosure is provided for the purpose of illustration, and it would be understood by a person with ordinary skill in the art that various changes and modifications may be made without changing technical conception and essential features of the present disclosure. Thus, it is clear that the above-described examples are illustrative in all aspects and do not limit the present disclosure. For example, each component described to be of a single type can be implemented in a distributed manner. Likewise, components described to be distributed can be implemented in a combined manner.

The scope of the present disclosure is defined by the following claims rather than by the detailed description of the embodiment. It shall be understood that all modifications and embodiments conceived from the meaning and scope of the claims and their equivalents are included in the scope of the present disclosure.

### EXPLANATION OF REFERENCE CHARACTERS

1: Blood pressure measuring device
100: First strap
130: First connector
200: Second strap
210: Air pocket
212: Compressing chamber
212-1: Expansion part
214: Auxiliary chamber
214-1: Concave part
220: Air flow path
300: Main body
310: Air supply unit
320: Measurement unit
330: Controller
340: Display unit
350: Manipulation unit
360: Power supply unit
370: Communication unit

We claim:

1. A blood pressure measuring device configured to be worn around a wrist of an examinee, comprising:
a first strap configured to be wound on a part of the wrist adjacent to an ulnar artery;
a second strap connected to the first strap to be worn around the wrist, configured to be worn on a part of the wrist adjacent to a radial artery, and including an air pocket into and from which air selectively flows; and
a main body provided on the first strap or the second strap and including a measurement unit configured to measure a pressure of the air pocket and a controller configured to calculate a blood pressure of a user based on the pressure of the air pocket measured by the measurement unit,
wherein the air pocket includes a compressing chamber which expands when filled with air and thus compresses the wrist and compresses the radial artery, and
the compressing chamber includes a first surface adjacent to the wrist and a second surface facing the first surface, and a part of the second surface protrudes toward the first surface and forms an expansion part.

2. The blood pressure measuring device of claim 1,
wherein the compressing chamber has one or more expansion parts.

3. The blood pressure measuring device of claim 1,
wherein the second strap has a plurality of holes formed along a longitudinal direction,
the air pocket is equipped with an air pocket ring, and
the air pocket ring is detachably coupled to any one of the plurality of holes to adjust a position of the air pocket on the second strap.

4. The blood pressure measuring device of claim 1,
wherein the main body includes a display unit configured to display the calculated blood pressure.

5. The blood pressure measuring device of claim 1,
wherein the main body includes a communication unit configured to transmit and receive data to and from an external device.

6. A blood pressure measuring device configured to be worn around a wrist of an examinee, comprising:
a first strap configured to be wound on a part of the wrist adjacent to an ulnar artery;
a second strap connected to the first strap to be worn around the wrist, configured to be worn on a part of the wrist adjacent to a radial artery, and including an air pocket into and from which air selectively flows; and
a main body provided on the first strap or the second strap and including a measurement unit configured to measure a pressure of the air pocket and a controller configured to calculate a blood pressure of a user based on the pressure of the air pocket measured by the measurement unit,
wherein the air pocket includes:
a compressing chamber which includes a first surface adjacent to the wrist and a second surface facing the first surface and expands when filled with air and thus compresses the wrist with the first surface and compresses the radial artery; and
an auxiliary chamber which communicates with a part of the second surface of the compressing chamber and expands when filled with air, and has a cross-sectional diameter that increases and decreases in a direction parallel to the first surface of the compressing chamber and thus forms a concave part.

7. The blood pressure measuring device of claim 6,
wherein the auxiliary chamber has one or more concave parts.
